(19) United States
(12) Patent Application Publication
(10) Pub. No.: US 2007/0179368 A1
(43) Pub. Date: Aug. 2, 2007

(54) Method of recognizing abnormal tissue using the detection of early increase in microvascular blood content

(75) Inventors: Vadim Backman, Chicago, IL (US); Hemant Roy, Highland Park, IL (US); Ramesh Wali, Darien, IL (US); Young L. Kim, Skokie, IL (US); Michael Siegel, Seattle, WA (US); Vladimir Turzhitsky, Evanston, IL (US)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignees: Northwestern University, Evanston, IL (US); Evanston Northwestern Healthcare, Evanston, IL (US)

(21) Appl. No.: 11/604,653

(22) Filed: Nov. 27, 2006

Related U.S. Application Data
(63) Continuation-in-part of application No. 11/261,452, filed on Oct. 27, 2005.
(60) Provisional application No. 60/801,947, filed on May 19, 2006.

(51) Int. Cl. A61B 5/00

(52) U.S. Cl. 600/315

(57) ABSTRACT

The present invention, in one aspect, relates to a method for examining a target for tumors or lesions using what is referred to as “Early Increase in microvascular Blood Supply” (EIBS) that exists in tissues that are close to, but are not themselves, the abnormal tissue and in tissues that precede the development of such lesions or tumors. While the abnormal tissue can be a lesion or tumor, the abnormal tissue can also be tissue that precedes formation of a lesion or tumor, such as a precancerous adenoma, aberrant crypt foci, tissues that precede the development of dysplastic lesions that themselves do not yet exhibit dysplastic phenotype, and tissues in the vicinity of these lesions or pre-dysplastic tissues.

### PRIORITY CLAIM

This application claims priority to U.S. Application No. 60/801,947 entitled “Guide-To-Colonoscopy By Optical Detection Of Colonic Micro-Circulation And Applications Of Same”, which was filed on May 19, 2006, the contents of which are expressly incorporated by reference herein. This application is also a continuation-in-part and claims priority to copending U.S. patent application Ser. No. 11/261,452 entitled “Multi-Dimensional Elastic Light Scattering”, filed Oct. 27, 2005 with the same assignee as the present invention, the disclosure of which is incorporated in its entirety herein by reference.

Some references, which may include patents, patent applications and various publications, are cited and discussed in the description of this invention The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### STATEMENT AS TO RIGHTS UNDER FEDERALLY-SPONSORED RESEARCH

This invention was made with Government support under Grant Nos. R01CA109861 and U01CA111257 awarded by the National Institutes of Health of the United States. Accordingly, the United States Government may have certain rights in this invention pursuant to the grant.

### FIELD OF THE INVENTION

The present invention relates generally to light scattering and absorption, and in particular to methods of recognizing possibly abnormal living tissue using a detected early increase in microvascular blood supply and corresponding applications including in vivo tumor imaging, screening, detecting and treatment, and, in particular, “Early Increase in microvascular Blood Supply” (EIBS) that exists in tissues that are close to, but are not themselves, the lesion or tumor and in tissues that precede the development of such lesions or tumors.

### BACKGROUND OF THE INVENTION

There are various techniques known for determining abnormality in tissues. Of these techniques, those that are most relevant to the present invention are techniques in which there is detected an increase in blood within tissue that is abnormal. While such techniques have advantages in and of themselves as compared to other methods, they require testing of the abnormal tissue itself, which may be difficult to detect. Further, such methods are usable only after the abnormality is sufficiently large, such as a cancerous tissue.

Accordingly, the present invention provides a variety of advantageous optical techniques for assisting in the detection of abnormal tissue, particularly using optical measurements, early in the development of the abnormal tissues themselves.

### SUMMARY OF THE INVENTION

The present invention, in one aspect, relates to a method for examining a target for tumors or lesions using what is referred to as “Early Increase in microvascular Blood Supply” (EIBS) that exists in tissues that are close to, but are not themselves, the abnormal tissue and in tissues that precede the development of such lesions or tumors. While the abnormal tissue can be a lesion or tumor, the abnormal tissue can also be tissue that precedes formation of a lesion or tumor, such as a precancerous adenoma, aberrant crypt foci, tissues that precede the development of dysplastic lesions that themselves do not yet exhibit dysplastic phenotype, and tissues in the vicinity of these lesions or pre-dysplastic tissues.

A particular application described herein is for detection of such lesions in colonic mucosa in early colorectal cancer, but other applications are described as well.

In one aspect, the present invention describes a method of providing an indication that living tissue within an organ of a body may be abnormal that includes identifying tissue of the organ that contains microvasulature therein, wherein the tissue does not contain the living tissue that may be abnormal and determining from the blood content within the microvasculature whether an early increase in microvascular blood supply exists in the tissue to indicate whether the living tissue or nearby tissue may be abnormal.

In another aspect, the present invention provides a method of providing an indication that living tissue within an organ of a body may be abnormal comprising the steps of:

inserting an illumination probe such that a light source within the illumination probe is disposed in a location that is at a surface of the organ;

illuminating microvasculature within a tissue of the organ, which tissue does not contain the living tissue that may be abnormal;

detecting scattered light that results from the step of illuminating the location, wherein the detected light is obtained substantially from light scattered from blood in the microvasculature that is within the tissue of the organ that does not contain the living tissue that may be abnormal;

estimating at least one of blood content and blood flow in the microvasculature using the detected light; and

obtaining the indication that the living tissue may be abnormal using the at least one of estimated blood content and blood flow.

In a further aspect, the steps of illuminating, detecting, and estimating are repeated for a plurality of different locations along the surface and a directional indication of a location of the abnormal living tissue is provided based upon at least some of the plurality of different locations.

In another aspect, the indication is used to decide when to perform another test to determine whether the living tissue within the organ may be abnormal.

### BRIEF DESCRIPTION OF THE DRAWINGS

These and other aspects and features of the present invention will become apparent to those of ordinary skill in the art upon review of the following description of specific embodiments of the invention in conjunction with the accompanying figures, wherein:

FIG. 1 illustrates organization of blood supply in colonic mucosa and submucosa.

FIG. 2 shows schematically according to one embodiment of the present invention a fiber-optic polarization-gated probe: (a) side view and (b) distal (i.e., close to tissue surface) tip.

FIG. 3 shows according to one embodiment of the present invention photographically a polarization-gated probe in an accessory channel of an endoscope.

FIG. 4 shows according to one embodiment of the present invention accuracy of optical measurement of Hb content in (a) superficial tissue (obtained from ΔI(k) using Eq.1) and in (b-d) subsuperficial tissue obtained using Equation (1) (panel (a)) and alternate methods: absorption band area (panel (b)) and absorption band intensity (panel (c)). Is' stands for transport mean free path length. The dashed line shows what the data would look like if the accuracy of measurements were 100%.

FIG. 5 shows alterations of blood supply in early experimental carcinogenesis observed using polarization-gated signal according to one embodiment of the present invention. The data shows EIBS in histologically normal mucosa (i.e., superficial tissue compartment) of AOM-treated rats two weeks after initiation of carcinogenesis by means of AOM injection. This early time point precedes the development of adenomas, aberrant crypt foci and any other currently known markers of colon carcinogenesis. EIBS was observed only in the distal colon of AOM-treated rats and no blood content increase was found in the proximal colon, consistent with the fact that precancerous and cancerous lesions develop primarily in the distal colon in this model.

FIGS. 6(a)-6(d) show schematically according to one embodiment of the present invention the observation of EIBS in our in vivo studies (n=196 patients). The x-axis shows a location of EIBS reading in relation to the location of an adenoma. Normal control values were taken from patients with negative colonoscopy from the same colonic segments where adenomas were found in patients with positive colonoscopy. (a) EIBS from total blood content; (b) EIBS in superficial tissue (e.g. mucosa) extends >30 cm, i.e. EIBS can be observed in colonic segments other than the one where an adenoma is located; (c) EIBS in subsuperficial tissue (e.g. mucosa and superficial mucosa) extends <30 cm from the location of an adenoma; (d) Benign, hyperplastic polyps do not lead to EIBS outside extend of a polyp.

FIG. 7 shows the data confirming the phenomenon of EIBS in AOM-treated rats by Western blot. Although Western blot clearly shows EIBS 8 weeks after initiation of carcinogenesis (i.e., aberrant crypt foci (ACF), pre-adenoma stage of colon carcinogenesis), the sensitivity and accuracy of Western blot was not sufficient to measure EIBS in pre-ACF and stage (two weeks after initiation of carcinogenesis). For comparison, the disclosed optics approach has sensitivity sufficient enough to detect EIBS at this earliest time point.

FIG. 8 shows according to one embodiment of the present invention a methodology of EIBS-assisted colonoscopy.

### DETAILED DESCRIPTION OF THE PREFERRED EMBODIMENTS

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views, As used in the description herein and throughout the claims that follow, the meaning of “a”, “an”, and “the” includes plural reference unless the context clearly dictates otherwise Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, not is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The present invention, in one aspect, relates to a method for examining a target for tumors or lesions using what is referred to as “Early Increase in microvascular Blood Supply” (EIBS) that exists in tissues that are close to, but are not themselves, the lesion or tumor. While the abnormal tissue can be a lesion or tumor, the abnormal tissue can also be tissue that precedes formation of a lesion or tumor, such as a precancerous adenoma, aberrant crypt foci, tissues that precede the development of dysplastic lesions that themselves do not yet exhibit dysplastic phenotype, and tissues in the vicinity of these lesions or pre-dysplastic tissues.

A particular application described herein is for detection of such lesions in colonic mucosa in early colorectal cancer (“CRC”), but other applications are described as well.

The target is a sample related to a living subject such as a human being or animal. The sample may be a part of the living subject such that the sample is a biological sample, wherein the biological sample may have tissue developing a cancerous disease.

The neoplastic disease is a process that leads to a tumor or lesion, wherein the tumor or lesion is an abnormal living tissue (either premalignant or cancerous), such as pancreatic cancer, a colon cancer, an adenomatous polyp of the colon, a liver cancer, a lung cancer, a breast cancer, or other cancers.

The measuring step is preferably performed in vivo, though it can be performed ex vivo as well. The measuring step may further comprise the step of acquiring an image of the target. The image, obtained at the time of detection, can be used to later analyze the extent of the tumor, as well as its location. Measuring of blood content using interacted light, which can include scattering as well as other optical methods, can include insertion of a probe for in-vivo usages in which blood content and/or flow is measured in tissue of a solid organ. Also, the present invention can be used to insert a probe into a body cavity, such as for measurements of tissues that are in the GI tract, respiratory track, or the like.

In one embodiment, the method comprises projecting a beam of light to a target that has tissues with blood circulation therein. At least one spectrum of light scattered from the target is then measured, and blood supply information related to the target is obtained from the measured at least one spectrum. The obtained blood supply information comprises data related to at least one of blood content, blood oxygenation, blood flow and blood volume.

The method according to an embodiment of the invention may include obtaining a first set of the blood supply information from a first location of the target and then obtaining a second set of the blood supply information from a second location of the target. The first set of the blood supply information at a first location of the target and the second set of the blood supply information at a second location of the target can then be compared to determine the status of the target. One can compare the data to indicate whether the tumor or lesion exists at all by comparison to previously established microvascular blood content values from patients who harbor neoplasia and from those who are neoplasia free. The data can also indicate whether the tumor or lesion is closer the first or second location by comparison of the blood content values from the first and second locations. Rather than measuring blood content in a given tissue site, at least two but preferably more tissue sites may be assessed located within a given area of tissue and the statistical properties of blood content or blood flow distribution can be determined for this area to determine the status of the target. For example, the maximal blood content within an area can be used to determine the status of the target. Other statistical measures include mean, average, median, standard deviation, maximal value, and minimal value. Rather than having different locations, the same location can be compared at different times, days, months or years apart, to determine the status of the target, and in particular whether the tumor or lesion has developed or if it previously existed whether it has gotten larger.

The present invention, in another aspect, relates to an apparatus for examining a target. In one embodiment, the apparatus comprises a light source configured and positioned to project a beam of light to a target; and means for measuring at least one spectrum of light scattered from the target; and means for obtaining blood supply information related to the target from the measured at least one spectrum.

The apparatus may further comprise a detector that obtains a first set of the blood supply information at a first location of the target. The same detector can be used to obtain a second set of the blood supply information at a second location of the target. An algorithm, which is executed by a controller or computer, analyzes that data is used to determine the status of the target, typically by comparing the first set of the blood supply information at a first location of the target and the second set of the blood supply information at a second location, although comparisons against reference blood supply information (that does or does not suggest the presence of a tumor or lesion) can be used as well. This same apparatus can be used to implement the method mentioned above where the same location is sampled at different points in time.

A superficial and subsuperficial polarization and spectral data analysis algorithm that allows for discrimination between spectral data obtained from the mucosal and the submucosal tissue is described in the following Equation 1. It is noted that this algorithm, because it is based on the quantitative analysis of spectroscopic signals recorded as a result of elastic scattering and absorption of light in tissue without the need for tissue biopsy or any other preparation, allows for almost real-time processing of the polarization gated signals, which makes it very useful for clinical screening applications.

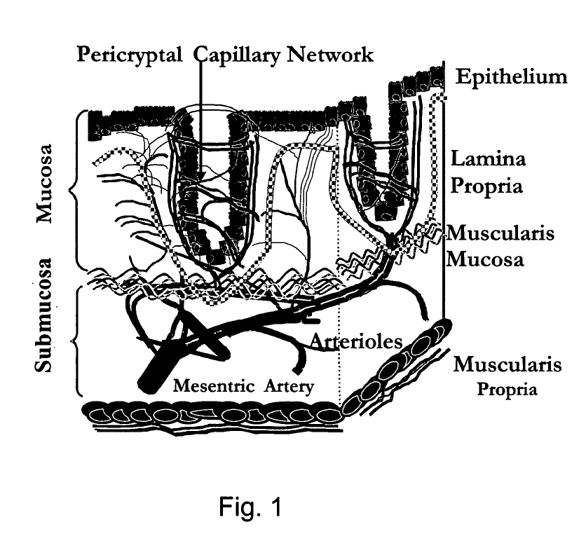

Polarization gating enables assessment of blood content in several tissue compartments at the same time. The two principal tissue compartments that are being analyzed for the blood content are the “superficial” (e.g. mucosal) and “subsuperficial” (e.g. mucosal and submucosal) tissues (FIG. 1). In order to assess blood content and/or blood flow in superficial tissue, polarization-gated spectrum is preferably used. Blood content in subsuperficial tissue can be measured by using co-polarized spectrum, arbitrarily polarized (also referred to as unpolarized or total) spectrum (which is the sum of co-polarized and cross-polarized signals), or cross-polarized spectrum. These three signals have progressively deeper depths of penetrations. The depth of penetration of these signals can be selected by the instrumentation design in order to selectively probe mucosal and mucosal/submucosal tissue for a given organ and tissue type. The polarization-gated signal S(λ) is taken as the difference between copolarized and crosspolarized signals, each normalized by the corresponding copolarized and crosspolarized spectra from a polytetrafluoroethylene reflectance standard (Ocean Optics). Co-polarized, arbitrarily polarized (also known as unpolarized) and cross-polarized signals (D(λ)) are normalized accordingly. In the teachings that follow, as an example, it is considered that subsuperficial blood content is measured from the cross-polarized signal. It should be understood, however, that similar analysis can be performed based on co-polarized and arbitrarily polarized signals. Thus, in principle, blood content can be measured for four different depths of penetration.

In both cases of superficial and subsuperficial blood content, it is assumed that the variability in path length due to differences in optical properties within the sample is small. While it is known that Beer's law cannot be directly applied to the analysis of scattered light because of unknown attenuation due to scattering, Beer's law served as the starting point for the analysis, as attenuation due to absorption has an inverse exponential relationship with absorber concentration. This assumption can be expressed as follows:
S(λ)=S_{scattering}(λ)exp[−L_{S}(α_{HbO2}A^{HbO2}(λ)+α_{Hb}A^{Hb}(λ))]
D(λ)=D_{scattering}(λ)exp[−L_{D}(β_{HbO2}A^{HbO2}(λ)+β_{Hb}A^{Hb}(λ))], (1)
where S_{scattering}(λ) and D_{scattering}(λ) represent light scattering signals from the superficial and subsuperficial layers of a sample, respectively, if it were devoid of absorbers. A(λ) represents the absorption spectrum of all of the absorbers present (oxygenated and deoxygenated hemoglobin), coefficients L_{S} and L_{D} represent the path lengths for polarization-gated and cross-polarized signals, and coefficients α and β represent the absorbers' concentrations for superficial and subsuperficial tissue depths. To account for different contributions of oxygenated and deoxygenated hemoglobin, two coefficients are used: α_{HbO2} and α^{Hb} in case of superficial blood content and β_{HbO2} and β_{Hb} in case of subsuperficial blood content. Similar analysis can be performed for co-polarized and arbitrarily polarized (i.e., total, unpolarized) spectra.

Spectra for deoxygenated and oxygenated blood content can be measured in a hemoglobin solution in water. The solution is placed in a glass-bottomed culture slide directly on top of a reflectance standard and measured to obtain a spectrum A^{HbO2}(λ). It is then deoxygenated by adding sodium dithionite to measure the A^{Hb}(λ). L_{S} and L_{D} are determined in the process of initial instrument calibration in tissue models. The remaining unknowns for the analysis are S_{scattering}(λ) and D_{scattering}(λ). To fill this gap it is assumed that expected S_{scattering}(λ) and D_{scattering}(λ) should have a smooth decreasing spectral line shape between λ=480 and 680 nm, thereby lacking spectral features of hemoglobin absorption, which include absorption bands at 542 and 576 nm in the case of oxygenated blood and 555 nm in the case of deoxygenated blood. In particular, second-order decreasing polynomial or inverse power-law spectral line shapes can be used as target line shapes with essentially the same results. Over this narrow spectral range, this assumption is reasonable. Thus the superficial and subsuperficial polarization and spectral data analysis algorithm tests values of α_{HbO2} and α_{Hb} for superficial and β_{HbO2}, and β_{Hb} for subsuperficial tissue over the range of interest and finds those that provide the best agreement between the resulting S_{scattering}(λ) and D_{scattering}(λ) and a target line shape in the least-squares sense. This process can be continued iteratively.

Once coefficients α_{HbO2} and α_{Hb} are found, a number of other related metrics characterizing blood content can be found including
total blood content=α_{HbO2}+α_{Hb} and
oxygen saturation=α_{HbO2}/(α_{HbO2}+α_{Hb}). (2)

The validity of all equations was verified using tissue phantom experiments.

In one embodiment, at least one spectrum of light scattered from the target is measured by a fiber optic probe, wherein the fiber optic probe comprises a polarization-gated fiber optic probe configured to detect the blood supply information. The light source comprises an incoherent light source (such as a xenon lamp).

In one embodiment, the fiber optic probe includes a proximal end portion, an opposite, distal portion, and a body portion with a longitudinal axis defined between the proximal end portion and the distal portion. The body portion is formed with a cavity along the longitudinal axis. At least one first type of fiber is used for delivering a beam of energy to a target, wherein the at least one first type fiber is at least partially positioned within the cavity of the body portion. An optical element is positioned at the proximal end portion and configured to focus the beam of energy to the target. At least one second type fiber is used for collecting scattered energy from the target, wherein the at least one second type fiber is at least partially positioned within the cavity of the body portion.

The fiber optic probe may further comprise at least one linear polarizer optically coupled to the at least one first type fiber and the at least one second type fiber and positioned proximate to the proximal end portion, and wherein the optical element is positioned at the proximal end portion and configured to focus the scattered energy from the target to the at least one linear polarizes for the at least one second type fiber to collect.

The optical element comprises at least one of a ball lens, a graded refractive index lens, an aspheric lens, cylindrical lens, convex-convex lens, and plano-convex lens, although preferably just a single lens is used. Lenses or any combinations of them other than these above-mentioned lenses can also be used. It is further noted that different lenses can be used to assist in discriminating measurements and to achieve different tissue penetration depths. Thus, for example, to achieve the shortest penetration depth, a lens can be positioned at the focal distance from the end of the light-collecting fibers with the fibers positioned symmetrically around the axis of the lens. This configuration further increases the intensity of collected light, particularly when a probe is at a distance form tissue, and provides improved stability of the signals collected by the probe in terms of different distances from tissue (if a probe is not in contact with tissue) and pressures exerted by the probe onto tissue (if a probe is in contact with tissue). Shorter penetration depth can also be achieved by using a lens with a shorter focal distance, smaller numerical aperture of the illumination and/or collection fibers, and larger distance between illumination and collection fiber. In principle, penetration depths from a few tens of microns to a few millimeters can be achieved by choosing a proper combination of these probe characteristics.

The at least one first type fiber comprises an illumination fiber, wherein the illumination fiber is optically coupled to the light source.

The at least one second type fiber can also be formed with one or more collection fibers, wherein the one or more collection fibers are optically coupled to an imaging spectrograph and a CCD at the distal end portion, which imaging spectrograph is used to obtain an image of the target. The body portion comprises a tubing.

The following further details of the preferred embodiments, will further describe the invention. As will become apparent, a substantial part of the following disclosure relates to the EIBS phenomenon as applied to determination of the presence of colonic neoplasia (adenomatous polyp or carcinoma) through analysis of colonic tissue remote to the lesion and as a guide to the determination of the location of a tumor or lesion with the colon. This disclosure is also applicable to detection of tumors or lesions within other organs, and to the extent variations exist with respect to such detection in different organs, such is noted.

Although it has been well established that blood supply to tumor tissue is increased, very little attention has been given to alterations in blood supply at the pre-neoplastic stage and histologically/endoscopically normal appearing mucosa outside the extend of a neoplastic or pre-neoplastic lesion, largely due to the methodological difficulties in reliably quantitating microvascular blood supply. EIBS is most pronounced in the very superficial mucosa (peri-cryptal capillary plexus). This makes up a very small amount of total colonic microcirculation. The reason polarization-gated optical spectroscopy can detect this is that it can specifically and accurately analyze this plexus.

It is further noted that the present invention can distinguish between benign and malignant tumors, such that when a tumor is seen, looking at the surrounding normal mucosa can assist in distinguishing a hyperplastic (benign) from an adenomatous (premalignant) tumor. Abnormal blood supply in the microscopically normal mucosa adjacent to the lesion—EIBS—will be seen in adenomatous but not hyperplastic polyps. Furthermore, previous angiogenesis studies focused on blood supply increase to a neoplastic lesion itself. EIBS manifests itself as an increase in blood supply in the microcirculation (primarily mucosa) supplying blood to epithelium. EIBS occurs very early during the process of colon carcinogenesis. Our data in animal models of colon carcinogenesis showed that EIBS starts at earlier than development of adenomas and aberrant crypt foci (i.e. the earliest marker of carcinogenesis) and precedes the development of currently known molecular markers of colonic neoplasia. Furthermore, EIBS can be detected outside a neoplastic lesion. This allows for detecting a lesion outside its physical extent, as discussed by the examples and further disclosure provided below.

### EXAMPLES

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

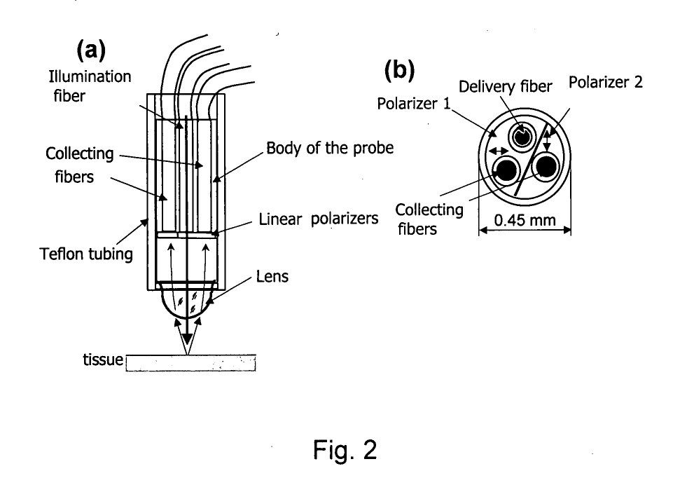

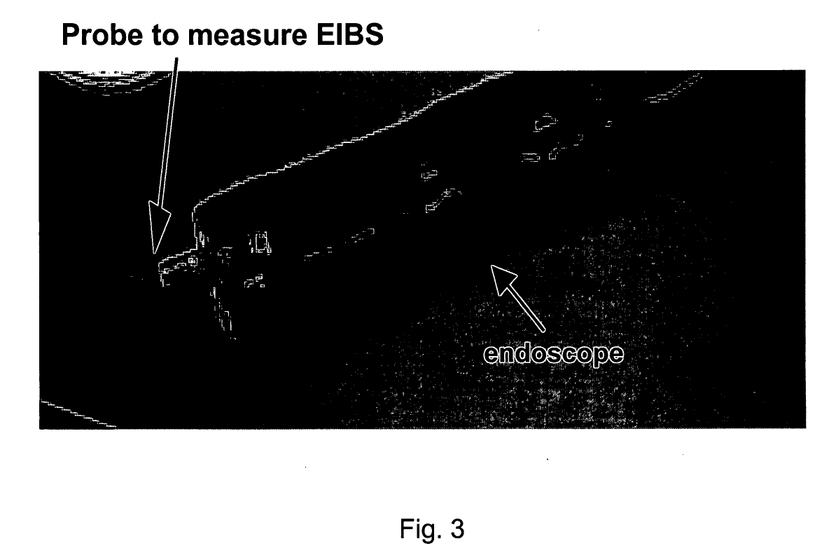

Polarization Gated Fiber-Optic Probe to Detect EIBS: In one aspect, a fiber-optic probe has been developed to accurately detect blood supply in tissue mucosa. FIG. 2 illustrates the design of the probe in one embodiment and FIG. 3 shows a photograph of the probe protruding from an accessory channel of a colonoscope. The probe has one or more 100 μm-diameter fibers, one of which was used for delivery of linearly polarized light from a Xe-lamp onto tissue surface and the other two fibers were for collecting scattered light from the tissue. A positive aspherical lens was positioned at the focal distance from the fiber tips. Several lens types were also tested, including ball, graded refractive index (GRIN), and aspherical lenses. All of the different types of lenses could be used and these provide different performance of the probe in terms of the depth of penetration. In the configuration where the lens was positioned at the focal distance from the fiber tips, it focused light backscattered from a sample onto different fibers depending on the angle of backscattering. It also ensured that all collection fibers receive scattered light from the same tissue site, which also coincides with the illumination spot. The lens does not have to be positioned at the focal distance from the fibers, but this configuration provides better performance in terms of 1) shorter penetration depth, in particularly for the polarization gated signal, 2) increases signal level and, thus, time required to collect the signal with sufficient signal-to-noise ratio, 3) prevents collection of specular reflection from probe and tissue surfaces, and 4) improves stability of the measurements in terms of probe displacement from tissue surface in non-contact geometry or the pressure exerted by the probe onto a sample. In the proximal end of the probe, the linear array of fibers was coupled to an imaging spectrograph and a CCD. Two thin film polarizer's were mounted on the proximal tip of the probe to polarize the incident light and enable collection of both polarization components (i.e. parallel I∥ and I⊥ perpendicular to the incident polarization) of the backscattered light to allow for polarization gating. All components of the probe were made from FDA approved materials.

A lens at the probe tip allows selecting a desired penetration depth. For example, to achieve a shorter penetration depth, a lens can be positioned at the focal distance from the end of the fibers with the fibers positioned symmetrically around the axis of the lens. Furthermore, one can use a lens with a shorter focal distance, smaller numerical aperture of the illumination and/or collection fibers, and a larger distance between the illumination and collection fiber. For example, probes were fabricated with a GRIN lens with the penetration depth in colon tissue for polarization-gated signal ˜85 microns (˜1.7 mean free path lengths) and that for cross-polarized light ˜260 microns. A ball lens probe with penetration depths ˜23 and 275 microns was also developed. As such, it is apparent that penetration depths from a few tens of microns to a few millimeters can be achieved by choosing a proper combination of these probe characteristics.

Polarization Gating: Polarization gating has been previously used to selectively record short-traveling photons as well as to increase contrast for photons emerging from deeper tissue. As has been shown by our group, the differential polarization signal ΔI (λ)=I∥(λ)−I⊥(λ) is primarily contributed by scatterers located close to the tissue surface and, therefore, particularly sensitive to the properties of the superficial tissues, e. g. epithelial. Our experiments showed that the contribution to the differential polarization signal from deeper tissue structures decreases exponentially with “optical distance” to the structure and, hence, with depth (τ=L/1s with L “physical” depth and 1s photon mean free path length in tissue). Because optical density of epithelium is much smaller than that of underlying connective tissue, in the colon, differential polarization signals are primarily collected from the epithelium plus up to ˜50 μm of underlying connective tissue. This near-surface portion of subepithelial stoma contains a network of capillaries supplying oxygen to the epithelium. Co-polarized signal I∥, arbitrarily polarized signal I∥+I⊥ and cross-polarized signal I⊥ contain information about progressively deeper tissue, up to several millimeters below the surface for certain probe configurations.

Measurement of Superficial Blood Content: The blood content in the capillaries immediately below epithelium can be quantitatively estimated from the spectral analysis of ΔI(A). We developed several methods of spectral data analysis. The following example [Eq.3] discusses an earlier version of the method that provides for analysis of superficial blood content. A more recent version [Eqs. 1 & 2] that provides improved accuracy of blood content estimation in both the superficial and subsuperficial mucosa is discussed above.

With respect to this earlier version, it operates based on a determination of blood content values that include effects of both scattering and absorption by red blood cells in the microvasculature and model the presence of absorption bands in the spectra due to both light absorption and scattering by blood.

We obtained the scattering images of rats' red blood cells (RBCs). Although Hb primarily absorbs visible light, it is not sufficient to measure only the absorption spectra of Hb molecules. RBCs, which are filled with Hb, are large scatterers approximately 7-8 microns in diameter. Therefore, the contribution from the RBCs couples both absorption and scattering. Our data demonstrate that differential polarization signal measured from a tissue ΔI(λ) can be written as
ΔI(λ)=ΔIs(λ)+αΔI_{RBC}, (3)
where ΔIs (λ) is the signal contributed by epithelial cells and other non-RBC components of the superficial tissue (not a priori known), ΔI_{RBC} is the signal experimentally measured from isolated ref blood cells (thus, this signal is known), and α is the number density of RBCs per mm². This early version of the polarization and spectral data analysis algorithm for superficial blood content was used to find the fitting parameter α by minimizing the Hb absorption bands in ΔIs (λ). This early version of the polarization and spectral data analysis algorithm, rather than using an exponential attenuation of light propagating in tissue by the tissue blood content that was used in Eq. 1, relies upon a linear calculation where the contribution from the red blood cells is assumed to be additive to that of tissue scattering; this contribution includes both light absorption and red blood cell scattering.

For in situ applications, where hemoglobin is present in both oxygenated (ΔI_{RBC-O2}(λ)), and deoxygenated (I_{RBC-O2}(λ)) forms,
ΔI_{RBC}(χ;λ)=χΔI_{RBC-O2}(λ)+(1−χ)I_{RBC-O2}(λ), (4)
with χ the oxygen saturation coefficient also determined by means of optimization.

Measurement of Subsuperficial Blood Content. We also assessed blood supply in the deeper tissue layers, i.e. mucosa and submucosa, via I⊥(λ) (as opposed to ΔI, this signal is primarily contributed not by single but multiple scattering process). For subsuperficial blood content, we developed several methods of spectral data analysis. The following example in Eq. 5 discusses an earlier version of the method. A more recent version that provides improved accuracy of blood content estimation is discussed above in Eqs. 1 & 2.

The changes in the blood supply to mucosa/submucosa is detected, as noted, by means of the analysis of the cross-polarized signal I⊥(λ). Briefly, a diffusion approximation model is fit to the data. The model I_{M} depends on the spectra of the transport scattering μ′ₛ (λ) and absorption coefficient
μ_{α}(λ)=χμ_{α-02}(λ)+(1−χ)χμ_{α-D02} (λ), (5)
which is contributed by both oxygenated μ_{α-02} and deoxygenated μ_{α-D02} Hb species with oxygen saturation χ found as a fitting parameter μ_{α} is proportional to the concentration of the respective form of Hb in tissue. It is conventionally assumed that Hb is the only significant absorber of visible light in the mucosa and μ′ₛ (λ) should not exhibit Hb absorption bands.

This diffusion approximation-based, early version of the algorithm requires substantial processing time (on the order of several minutes per sample), rather than the nearly real-time results from the algorithm of Eq. 1 discussed above. The reason for such an improvement is because the algorithm of Eq. 1 does not require the use of a diffusion approximation that is computationally intensive.

Measurement of Oxygen Saturation. As discussed above, due to distinctly different absorption spectra of oxy- and deoxy-hemoglobin, not only does spectral analysis of polarization gated signals enable measurement of blood content but also blood oxygenation (aka. oxygen saturation, S_{O2}=χ. We validated S_{O2} calculations from spectral data. The accuracy of oxygen saturation measurement was excellent with error <1%.

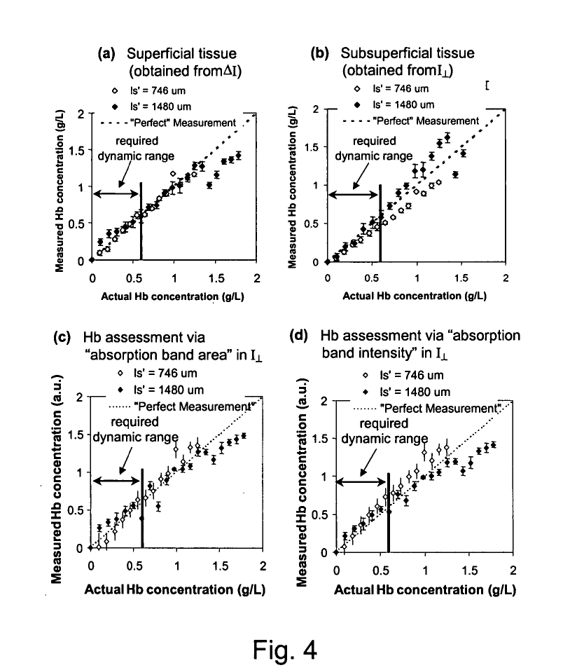

Accuracy of EIBS Assessment Using Fiber-Optic Probe. We also validated the ability of the probe to assess hemoglobin concentration in studies with tissue models. The tissue models were fabricated and the analysis of spectral data was performed as discussed above. As shown in FIG. 4, the probe enables accurate assessment of hemoglobin concentration. The standard error of measurements for concentrations <12 g/L for superficial tissue was <0.01 g/L and that for deeper tissue was <0.02 g/L. We point out that according to EIBS data in animals as well as humans, the dynamic range of Hb concentrations was well within this range. Thus, the probe provides sufficiently accurate measurement of blood content in physiologic range with error of measurement sufficient to reliably identify EIBS.

It is also possible to measure blood content based on the analysis of the area of hemoglobin absorption spectral band and/or the maximum of this absorption band. As shown in FIGS. 4(c,d), both “absorption band area” and “absorption band intensity” methods enable accurate assessment of hemoglobin concentration with the 10 error of measurements for concentrations <1.2 g/L 0.02 g/L and 0.03 g/L, respectively, and for concentrations from 1.2 to 18 g/L, 0.07 g/L and 0,09 g/L, respectively.

EIBS Precedes Formation of Known Markers of Colon Carcinogenesis. EIBS precedes the development of any currently known histologic or molecular markers of colon carcinogenesis.

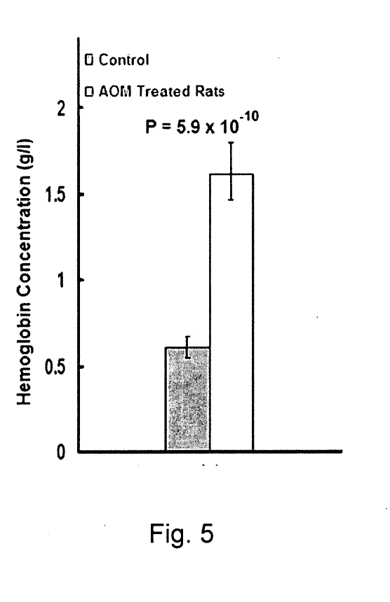

Specifically, we assessed blood content in the colons of rats treated with a colon specific carcinogen, azoxymethane (AOM). The AOM-treated rat model is one of the most robust and widely used animal models of CRC. As in humans, in this model, neoplasia progresses through a well defined sequence of events with the exception that the time course of carcinogenesis in the AOM-treated rat model is much faster than the one in humans: the earliest detectable marker of carcinogenesis, ACF, develops in 4-12 weeks after AOM injection, adenomas are observed in 20-30 weeks, and carcinomas develop after 40 weeks. No histologic, molecular or genetic markers have been shown to allow earlier diagnosis (<4-12 weeks). As shown in FIG. 5, our data demonstrate that EIBS occurs as early as 2 weeks after AOM-injection (p-value<10⁻⁹). Importantly, EIBS was detected only in the distal colon and not in the proximal colon (p-value<10⁻¹¹). This mirrors the progression of carcinogenesis in the AOM-treated rat model as AOM induces carcinogenesis primarily in the distal colon with only minimal effect on the proximal colon, as has been validated by numerous studies and our data as well.

EIBS Is an Accurate Predictor of Colonic Neoplasia: Animal Study. In order to assess whether EIBS may serve as a clinically useful biomarker, we determined the performance characteristics of EIBS to detect future ACF in AOM-treated rats. It was found that EIBS had excellent ability to distinguish animals at risk for CRC from the negative controls even at the pre-ACE stage of CRC, two weeks after AOM treatment. Indeed, the diagnostic accuracy of EIBS far exceeded conventional markers with high (>90%) sensitivity, specificity, positive and negative predictive values even at the earliest stages of colon carcinogenesis, preceding the development of currently known markers of CRC (Table 1).

TABLE 1
EIBS diagnosis of
predisposition to CRC in AOM-

[symbol]

| | |
|---|---|
| Sensitivity | 94% |
| Specificity | 96% |
| PPV | 97% |
| NPV | 92% |

EIBS Gradient Localizes Adenomas: In Vivo Clinical Study. To prove that EIBS and, importantly, EIBS gradient (i.e., progressive increase in blood content towards an adenoma) can be observed in vivo. A pilot investigation was conducted in human subjects undergoing screening colonoscopies (196 patients including 48 with adenomas out of which 43 were diminutive and 5 advanced, 27 subjects with hyperplastic benign polyps, and 121 patients with negative colonoscopies). We used an endoscopically compatible fiber-optic probe discussed in the preceding section. The probe was inserted into the accessory channel of a colonoscope. During colonoscopy, EIBS spectral data were acquired by the probe from the following locations: adenomatous polyp (if present), an endoscopically normal location within 10 cm from the adenoma, from the same colonic segment where the adenoma was located (typically within 30 cm from the adenoma) and the other segments (dubbed “outside” segments).

In patients with negative colonoscopy, measurements were taken at random from each of the three colonic segments (i.e., descending colon including rectum and sigmoid colon, mid-transverse colon, and ascending colon including the cecum). On average, three spectra were obtained from each tissue site and more than 10 different tissue sites were probed for each patient.

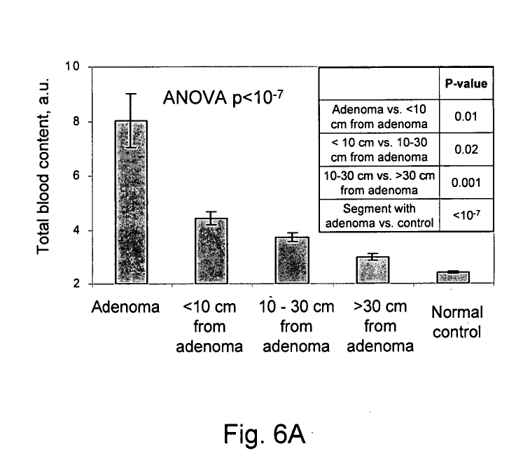

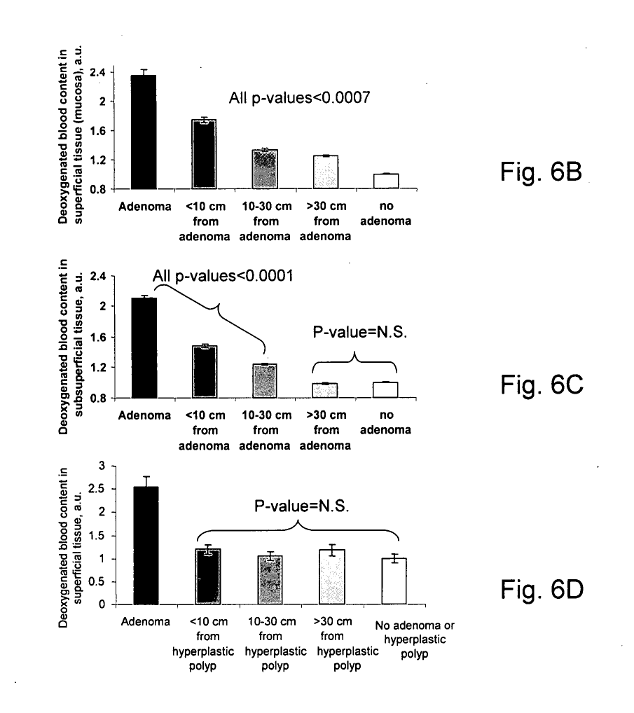

Our data (FIG. 6) demonstrate a marked augmentation of blood content in the uninvolved (endoscopically and histologically normal) colonic mucosal in patients with adenomas compared to the control subjects. Importantly, EIBS progressively increased when approaching a neoplastic lesion. Indeed, EIBS was noticeable about 30 cm from the location of the adenoma and progressed at 10 cm from the lesion and at the site of the lesion itself. It is this property of EIBS that may guide an endoscopist to identify high-risk colonic segments. EIBS in superficial tissue was observed even for locations >30 cm away from an adenoma (in colonic segments other than the one where the adenomas were found) (FIG. 6(a)). For comparison, EIBS in subsuperficial tissue was more localized and was observed only for distances <30 cm (FIG. 6(b)). This is consistent with our ex vivo data demonstrating that the spatial extend of EIBS decreases with tissue depth. Finally, FIG. 6(c) demonstrates that hyperplastic polyps do not result in EIBS outside their extend. This is also a promising result as it indicates that the absence of EIBS outside a polyp can be used to determine if a polyp is adenomatous or hyperplastic during the colonoscopy or other endoscopic procedure.

The performance characteristics of EIBS gradient to distinguish colonic segments with and without advanced adenomas are shown in Table 2. These characteristics are encouraging, particularly since because no other currently available technique enables sensing presence of adenomas by analysis of tissue outside the spatial extend of an adenoma. While characterizing the age of an adenoma is not possible, factoring the age of the person may be useful since as one ages microvascular blood content goes down in controls (neoplasia free).

TABLE 2
EIBS localization of adenomas in
humans by in vivo assessment of EIBS 10-30 cm

| | |
|---|---|
| Sensitivity | 100% |
| Specificity | 70% |

The performance characteristics of EIBS gradient to distinguish colonic segments with and without adenomas (as compared to advanced adenoma's above), as well as to differentiate between a tissue site located within 10 cm from the adenoma and between 10 and 30 cm, are shown in Table 3 below.

TABLE 3

| | Sensitivity | Specificity | PPV | NPV |
|---|---|---|---|---|
| <10 cm vs. | 95% | 68% | 72% | 96% |
| 10-30 cm from | | | | |
| adenoma | | | | |
| Segment | 89% | 79% | 87% | 80% |
| with adenoma vs. | | | | |
| normal control | | | | |
| Adenoma | 97% | 92% | 89% | 96% |
| vs. normal control | | | | |

The diagnostic performance of EIBS is also superior to conventional CRC screening techniques. For instance, a recent study demonstrated that FOBT and fecal DNA analysis had a sensitivity of 10.8% and 18.2%, respectively, and the sensitivity and positive predictive value of flexible sigmoidoscopy was reported to be only 52% and 6%, respectively. Furthermore, the analysis of our in vivo data showed that there was minor variation in microvascular blood content among the three colonic segments in control subjects, males vs. females, and patients of different age (from 40 to 80 years old). The accuracy of EIBS-based colonoscopy guidance may be improved by accounting for these variations.

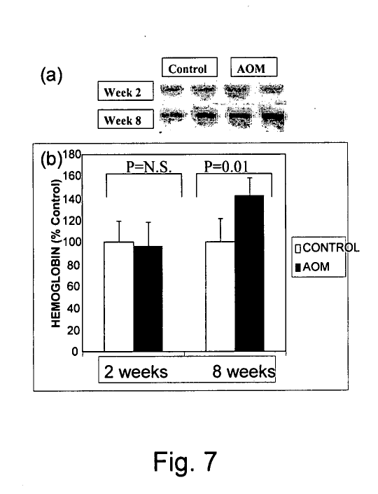

Non-optics Confirmation of EIBS. We also wanted to confirm EIBS by use of a non-optics methodology. We used Western blotting and evaluated blood content in the mucosa/submucosa in AOM-treated and control rats. The distal colons of AOM-treated and age-matched control rats were gently scraped. Mucosal homogenates were made and 25 μg of protein was separated on a 10% SDS-PAGE gel, transferred to PDVF membranes and blocked with 5% non-fat milk. Membranes were probed with a polyclonal antibody to hemoglobin (1:300 dilution/overnight at 4C, Santa Cruz Biotechnology) and xerograms were developed with enhanced chemiluminescence and quantitated with a laser densitometer. One clear band at the appropriate molecular weight was noted (68 kDa) as shown in FIG. 7(a). This band did not appear on any negative controls (including lysates of two colon cancer cell lines HT-29 and HCT-116 and rat samples probed with secondary antibody alone). Quantitation of the immunoblot analysis (relative to age-matched controls) is demonstrated in FIG. 7(b). As can be seen, there is a significant increase in hemoglobin content in the distal colon at 8 weeks (p=0.01). Blots were stripped and probed for β-actin to confirm uniform protein loading (data not shown).

This provides critical non-optics corroboration of EIBS. Moreover, it underscores the relative lack of sensitivity of non-optics based technologies. For instance, while the 8-week data are significant, the increase is much less dramatic than the 3-fold augmentation of EIBS noted with spectroscopy. Moreover, mucosal blood content analysis failed to reveal a difference at 2 weeks despite highly significant changes seen with spectroscopic analysis. We believe that this dramatic sensitivity of spectroscopic analysis is related in part to its ability to precisely assay just the vasculature. Mucosal scrapings, on the other hand, no matter how gentle, probably samples some of the larger blood vessels in the submucosa. Since we believe that EIBS in the histologically normal mucosa is primarily a microvascular phenomenon, assaying the deeper larger blood vessels can easily obscure the subtle early changes in the microvasculature.

Further Applications of EIBS

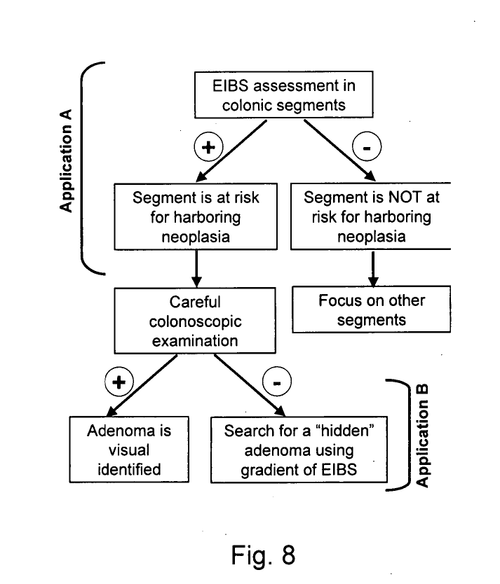

Using 4D-ELF or 2D-ELF (as described hereinafter) and polarization gated spectroscopy, this EIBS biological phenomenon in colonic mucosa in early CRC can be used for early stage detection of lesions. Importantly, spatially, EIBS extended outside the location of a neoplastic lesion (within at least ˜⅓ of colon from the lesion and beyond, depending on the depth of tissue) and its magnitude increased in the proximity to adenomas. Thus, our data showed that EIBS allowed remarkably accurate determination as to whether a given colonic segment harbors adenoma and could be used to indicate to an endoscopist the proximity of an adenoma. The methodology is to use EIBS to reduce colonoscopic miss rate (15-20% for adenomas and 6-12% for advanced adenomas) by guiding colonoscopy. We propose various applications of EIBS, two of which are illustrated as Application A and Application B in FIG. 8.

Application A: EIBS assessed from a given colonic segment will signal endoscopist that this segment is at risk for harboring adenomas and requires more rigorous colonoscopic evaluation. If a segment is not at-risk as determined by EIBS measurements, an endoscopist may make decision to focus on other colonic segments that may require more intense examination.

Application B: If adenoma is not readily visualized within this segment, increase magnitude of EIBS with approaching a lesion will guide an endoscopist in search for a hidden neopalsia.

Other usages of EIBS, Application C: EIBS can be assessed from distal colon during flexible sigmoidoscopy to assist in detection of the presence of adenomas and tumors in the proximal colon. As is known, a sigmoidoscopy is similar to a colonoscopy but examines only the lower colon and rectum. In such a procedure, the EIBS probe can be used in addition to the sigmoidoscopy probe to obtain the EIBS data. Furthermore, EIBS can be assessed from the rectum either via flexible sigmoidoscopy, a stand-alone fiber-optic probe, or a probe as part of an endoscopic device to assist in detection of the presence of adenomas and tumors in the other parts of colon.

Other usages of EIBS, Application D: EIBS can be assessed during colonoscopy, flexible sigmoidoscopy, or other endoscopic procedures to predict the development of future precancerous or cancerous lesions and, thus, assist in determining the schedule (e.g., frequency and time intervals) of future colonoscopies or flexible sigmoidoscopy procedures for a given patient.

Other usages of EIBS, in addition to the usages described above are within the intended scope of the invention, and particularly in conjunction with other diagnostic methods.

In addition to 4D-ELF and polarization gated spectroscopy, other spectroscopic techniques such as, 2D-ELF, enhanced backscattering and low-coherence enhanced backscattering (LEBS) spectroscopy, and OCT can also be used to practice the present invention.

For example, EIBS can be used in conjunction with a screening colonoscopy, in which case the EIBS probe can be used in addition to the colonoscopy probe to obtain the EIBS data.

The foregoing description of the exemplary embodiments of the invention has been presented only for the purposes of illustration and description and is not intended to be exhaustive or to limit the invention to the precise forms disclosed. Many modifications and variations are possible in light of the above teachings.

What is claimed:

1. A method of providing an indication that living tissue of a body may be abnormal comprising the steps of:
inserting an illumination probe such that a light source within the illumination probe is disposed in a location that is at a surface of an organ;
illuminating, at the location, tissue of the organ and microvasculature therein with light from the light source that is emitted from the illumination probe, wherein the tissue that is illuminated with the light does not contain the living tissue that may be abnormal;
detecting interacted light that results from the step of illuminating the tissue as detected data, wherein the interacted light is obtained substantially from the light that then interacts with blood in the microvasculature that is within the tissue of the organ, which tissue does not contain the living tissue that may be abnormal;
estimating at least one of blood content and blood flow in the microvasculature using the detected data; and
obtaining the indication that the living tissue may be abnormal using the at least one of estimated blood content and blood flow, the step of obtaining including the step of determining whether there exists an increase in the at least one of estimated blood content and blood flow in the microvasculature.

2. The method according to claim 1 wherein the organ contains the living tissue for which the indication is being obtained.

3. The method according to claim 1 wherein another organ contains the living tissue for which the indication is being obtained, and the another organ is different from the organ containing the tissue that is illuminated in the step of illuminating.

4. The method according to claim 1 wherein the interacted light in the step of detecting is obtained from the light that is scattered by the blood in the microvasculature.

5. The method according to claim 1 wherein the interacted light in the step of detecting is obtained from the light that is scattered and absorbed by the blood in the microvasculature.

6. The method according to claim 1 wherein the interacted light in the step of detecting is obtained from the light that is absorbed by the blood in the microvasculature.

7. The method according to claim 1 wherein the living tissue is a precancerous living tissue that may be abnormal.

8. The method according to claim 7 wherein the precancerous living tissue that may be abnormal is a predysplastic stage tissue.

9. The method according to claim 8 wherein the predysplastic stage tissue is at least one of histologically normal, macroscopically normal, and endoscopically normal.

10. The method according to claim 1 wherein the step of inserting the illumination probe inserts the probe into the organ, such that the surface of the organ is an inner surface of the organ.

11. The method according to claim 10 wherein the organ is the colon.

12. The method according to claim 10 wherein the organ is one of the stomach, the duodenum, the bladder, the esophagus, the oral cavity and the lung.

13. The method according to claim 10 wherein the organ is one of the uterus, the urethra and the prostate.

14. The method according to claim 1 wherein the step of detecting detects at least one of the following components of the interacted light: co-polarized, cross-polarized, and unpolarized interacted light.

15. The method according to claim 14 wherein the step of estimating estimates blood content.

16. The method according to claim 15 wherein the step of estimating the blood content estimates a concentration of red blood cells.

17. The method according to claim 15 wherein the step of estimating the blood content estimates a concentration of hemoglobin.

18. The method according to claim 15 wherein the step of estimating the blood content estimates a concentration of de-oxygenated hemoglobin.

19. The method according to claim 15 wherein the step of estimating the blood content estimates a concentration of oxygenated hemoglobin.

20. The method according to claim 15 wherein the step of estimating the blood content estimates one of blood flow and a rate of blood flow.

21. The method according to claim 15 wherein the step of estimating the blood content estimates oxygen saturation in the blood.

22. The method according to claim 14 wherein the step of estimating one of the blood content and the blood flow estimates a statistic of blood content or blood flow with an area of the living tissue.

23. The method according to claim 22 wherein the statistic is one of mean, average, median, standard deviation, maximal value, and minimal value.

24. The method according to claim 14 wherein the step of detecting detects interacted light from the surface to a submucosal layer.

25. The method according to claim 14 wherein the step of detecting the tissue detects interacted light from the surface to a mucosal layer.

26. The method according to claim 1 wherein the steps of inserting, illuminating and detecting are performed during a same period of time when a screening colonoscopy is performed.

27. The method according to claim 1 wherein the steps of inserting, illuminating and detecting are performed during a same period of time when a sigmoidoscopy is performed.

28. The method according to claim 1 wherein the steps of inserting, illuminating and detecting are performed using a stand-alone probe.

29. The method according to claim 1 wherein the steps of inserting, illuminating and detecting are performed using a probe disposed at least partially within an endoscopic device.

30. The method according to claim 1 wherein the step of inserting the probe inserts the probe adjacent to the organ, such that the surface of the organ is an outer surface of the organ.

31. The method according to claim 30 wherein the step of inserting the probe inserts the probe into a small bowel, adjacent to a pancreas, to detect the abnormal living tissue in the pancreas.

32. The method according to claim 1 wherein the step of obtaining the indication includes the step of comparing the estimated blood content with a baseline blood content.

33. The method according to claim 32 further including the step of establishing the baseline blood content.

34. The method according to claim 33 further including the step of establishing the baseline blood content based upon measurements of blood content of a region surrounding the organ.

35. The method according to claim 33 further including the step of establishing the baseline blood content based upon measurements of blood content of a plurality of bodies other than the body.

36. The method according to claim 33 further including the step of establishing the baseline blood content based upon measurements of blood content of the body.

37. The method according to claim 1 wherein the step of estimating estimates blood content.

38. The method according to claim 37 wherein the step of estimating the blood content estimates a concentration of red blood cells.

39. The method according to claim 37 wherein the step of estimating the blood content estimates a concentration of hemoglobin.

40. The method according to claim 37 wherein the step of estimating the blood content estimates a concentration of de-oxygenated hemoglobin.

41. The method according to claim 37 wherein the step of estimating the blood content estimates a concentration of oxygenated hemoglobin.

42. The method according to claim 37 wherein the step of estimating the blood content estimates one of blood flow and a rate of blood flow.

43. The method according to claim 37 wherein the step of estimating the blood content estimates oxygen saturation in the blood.

44. The method according to claim 1 wherein the step of estimating one of the blood content and the blood flow estimates a statistic of blood content or blood flow with an area of the living tissue.

45. The method according to claim 44 wherein the statistic is one of mean, average, median, standard deviation, maximal value, and minimal value.

46. The method according to claim 1 wherein the indication from the step of obtaining indicates that the living tissue may be abnormal at a future point in time.

47. The method according to claim 1 wherein the steps of illuminating, detecting, and estimating are repeated for a plurality of different locations along the surface; and
wherein the step of obtaining provides a directional indication of a location of the living tissue that may be abnormal based upon at least some of the plurality of different locations.

48. The method according to claim 47 wherein the plurality of different locations are separated by a distance of greater than 5 cm.

49. The method according to claim 47 wherein the plurality of different locations are separated by a distance of greater than 10 cm.

50. The method according to claim 47 wherein the directional indication is provided by a gradient of the indication, in which the indication increases when within 10 cm of the location of abnormal tissue.

51. The method according to claim 50 wherein an area where the gradient is increased indicates an increased likelihood abnormal living tissue.

52. The method according to claim 47, wherein the steps of illuminating and detecting estimating are repeated a plurality of different times.

53. The method according to claim 52 wherein the plurality of different times occur during a single procedure, and the interacted light detected from each of the plurality of different times is normalized together to determine a normalized interacted light that is used in the step of estimating.

54. The method according to claim 47 wherein the step of obtaining the indication includes the step of comparing the estimated blood content with a baseline blood content.

55. The method according to claim 47 wherein the living tissue is a precancerous living tissue that may be abnormal.

56. The method according to claim 55 wherein the precancerous living tissue that may be abnormal is a predysplastic stage tissue.

57. The method according to claim 56 wherein the predysplastic stage tissue is at least one of histologically normal, macroscopically normal, and endoscopically normal.

58. The method according to claim 47 wherein the step of inserting the illumination probe inserts the probe into the organ, such that the surface of the organ is an inner surface of the organ.

59. The method according to claim 58 wherein the organ is the colon.

60. The method according to claim 1 further including the step of using the indication to decide when to perform another test to re-determine whether the living tissue within the organ may be abnormal.

61. The method according to claim 60 wherein the step of using determines when to perform another test using periods of months.

62. The method according to claim 60 wherein the step of using determines when to perform another test using periods of years.

63. The method according to claim 1 wherein the step of estimating uses a polarization and spectral data analysis algorithm.

64. The method according to claim 63 wherein the polarization and spectral data analysis algorithm is a superficial and subsuperficial polarization and spectral data analysis algorithm that estimates blood content.

65. The method according to claim 64 wherein the superficial and subsuperficial polarization and spectral data analysis algorithm is based on a determination of blood content values that show deviation from baseline blood content values that represent normal tissue that does not have an early increase in blood supply.

66. The method according to claim 65 wherein the deviation is recognized by comparing a baseline scattering spectrum that shows the baseline blood content with a spectrum obtained from the detected data using a least squares analysis.

67. The method according to claim 65 wherein the superficial and subsuperficial polarization and spectral data analysis algorithm accounts for exponential attenuation of the interacted light due to presence of the blood in the microvasculature as a function of blood concentration.

68. The method according to claim 64 wherein the step of estimating includes modeling the detected data as a monotonic function of wavelength including at least one of the following: first-order, second-order, high-order polynomial, and an inverse power-law functions.

69. The method according to claim 68 wherein a model used in the step of modeling ignores an effect of blood absorption in the detected data, and wherein the detected data contains a spectrum of the interacted signal.

70. The method according to claim 64 wherein the step of estimating is performed in substantially real-time.

71. The method according to claim 63 wherein the polarization and spectral data analysis algorithm is a superficial polarization and spectral data analysis algorithm that estimates blood content.

72. The method according to claim 71 wherein the effect of red blood cells is modeled as additive to the rest of the scattering signal.

73. The method according to claim 72 wherein the spectrum of scattering signal without the effect of blood absorption is modeled as a monotonic function of wavelength including at least one of the following: first-order, second-order, high-order polynomial, and an inverse power-law functions.

74. The method according to claim 63 wherein the polarization and spectral data analysis algorithm is a subsuperficial polarization and spectral data analysis algorithm that estimates blood content.

75. The method according to claim 74 wherein the subsuperficial polarization and spectral data analysis algorithm includes a diffusion approximation.

76. The method according to claim 1 wherein the illuminated tissue is at least one of histologically normal, macroscopically normal, and endoscopically normal.

77. A method of providing an indication that living tissue within an organ of a body may be abnormal comprising the steps of:
identifying tissue of the organ that contains microvasulature therein, wherein the tissue does not contain the living tissue that may be abnormal; and
determining from the blood content within the microvasculature whether an early increase in microvascular blood supply exists in the tissue to indicate whether the living tissue may be abnormal.

78. The method according to claim 77 wherein the step of identifying tissue includes the step of obtaining a biopsy of the tissue.

79. The method according to claim 77 wherein the step of determining uses an optical detection to determine whether the early increase in microvascular blood supply exists.

80. The method according to claim 74 wherein the step of determining uses a non-optical detection to determine whether the early increase in microvascular blood supply exists.

81. The method according to claim 74 wherein the living tissue that may be abnormal is precancerous living tissue.

82. The method according to claim 81 wherein the precancerous living tissue that may be abnormal is a predysplastic stage tissue.

83. The method according to claim 82 wherein the predysplastic stage tissue is histologically normal and microscopically normal.
